(19) United States
(12) Patent Application Publication
(10) Pub. No.: US 2026/0090733 A1
(43) Pub. Date: Apr. 2, 2026

(54) MULTIPLE SENSOR ACOUSTIC RESPIRATORY MONITOR

(71) Applicant: Covidien LP, Mansfield, MA (US)

(72) Inventors: Mingxia SUN, Shanghai (CN); Zhenhua YUE, Shanghai (CN); Yi WU, Shanghai (CN); Yingying LIU, Shanghai (CN); Ling JI, Shanghai (CN)

(73) Assignee: Covidien LP, Mansfield, MA (US)

(21) Appl. No.: 19/114,451

(22) PCT Filed: Sep. 30, 2022
(86) PCT No.: PCT/CN2022/123375

§ 371 Date: Mar. 23, 2025

(51) Int. Cl. A61B 5/08; A61B 5/00; A61B 7/00

(52) U.S. Cl. CPC A61B 5/0816; A61B 5/6823; A61B 5/725; A61B 5/7264; A61B 5/7435; A61B 7/003

(57) ABSTRACT

An acoustic respiratory monitoring system (105) detects, monitors, and/or tracks acoustic features of respiratory conditions for a human patient. Acoustic respiratory data is derived from breathing sound captured by a sensor array (410) comprising a plurality of acoustic sensor elements (412). A plurality of logical channels is determined based on the acoustic respiratory data. An adventitious feature is detected in the breathing sound using the plurality of logical channels, and an indication of an abnormal respiratory sound is provided via a user interface (800), in response to detecting the adventitious feature. The sensor array (410) may be integrated with a wearable article (560), such as a shirt, vest, or belt, for example.

### BACKGROUND OF THE INVENTION

Airway diseases, such as asthma, emphysema, chronic obstructive pulmonary disease (COPD), and bronchiectasis, adversely affect the ability to breath due to inflammation or other conditions that hinder unrestricted airflow through a patient's airway to the lungs. The sounds produced by a patient during breathing play a substantial role in detecting and diagnosing the presence of airway diseases in a patient. For example, a physician often will use a stethoscope to ascertain sounds produced while the patient inhales and exhales. Typically, the patient is asked to breathe in and out deeply as the physician positions the stethoscope at various locations on the patient's chest and back and listens to the sounds produced by the patient's airway. In addition to the normal sounds of air movement, the physician may also be able to detect atypical sounds, such a crackles, as the patient breathes in (inspiration) and wheezes as the patient breathes out (expiration). Crackles and wheezing are just two examples of atypical breathing sounds that are often signs of an airway affected by disease. However, the effectiveness of a physician to recognize atypical sounds and detect a respiratory condition is subject to that physician's training and experience.

### SUMMARY OF THE INVENTION

The present disclosure is directed, in part, to multiple sensor based acoustic respiratory monitoring systems and methods, substantially as shown and/or described in connection with at least one of the figures, and as set forth more completely in the claims.

Systems and methods are disclosed related to acoustic respiratory monitoring technologies for detecting, monitoring, and tracking, audible symptoms of airway diseases, and for other purposes. In some embodiments, an acoustic respiratory monitoring system includes a sensor array that includes multiple respiratory sounds acquisition sensors, and a respiratory monitoring device that processes acoustic measurement data from the sensor array to evaluate respiratory sounds produced by a patient's breathing. In some embodiments, the respiratory monitoring system or device includes a user interface through which a healthcare professional can select, filter, and/or manipulate signals from the sensor array, and/or compare current breathing sound patterns to previously acquired breathing sound patterns from the patient. The respiratory monitoring device may also apply logic to correlate patient breathing sound patterns with known adventitious patterns for one or more particular airway diseases, and present predictions from those correlations to the healthcare professional. The respiratory monitoring device may record, visualize, or play respiratory sounds collected from the sensor array in real time. In some embodiments, the user interface includes functionally enabling the healthcare professional to selectively view and/or listen to real-time and/or previously processed breathing sound patterns or other information, and may further include functionality to selectively filter, process, or display acoustic measurement data from one or at least a portion of the respiratory sounds acquisition sensor elements.

The sensor array comprising the multiple sensor elements may be integrated, at least in part, with a wearable article, such as a shirt, vest, chest strap, or belt, for example. Arranging one or more of the sensor elements on a wearable article ensures that such sensor elements can be positioned in approximately the same position across a series of respiratory sounds acquisition sessions so trends in breathing sound patterns are more directly comparable. Arrangement of one or more of the sensor elements on a wearable article may also facilitate long-term monitoring or ambulatory monitoring of the patient as respiratory sounds information can be measured as the patient goes about their daily activities. The capture of acoustic respiratory data contemporaneously by multiple sensor elements distributed about the patient's body enables a set of data to be acquired and processed that comprise a diverse set of acoustic data for each inhale-exhale event. In this way, these embodiments can provide greater context for detecting and classifying adventitious patterns and tracking adventitious patterns over time.

### BRIEF DESCRIPTION OF THE DRAWING

The embodiments presented in this disclosure are described in detail below with reference to the attached drawing figures, wherein:

FIG. 1 is a block diagram illustrating an operating environment for an acoustic respiratory monitoring system, in accordance with embodiments of the present disclosure;

FIG. 2 is a block diagram illustrating an example respiratory monitor, in accordance with embodiments of the present disclosure;

FIG. 3 is a block diagram illustrating an example adventitious pattern correlation function, in accordance with embodiments of the present disclosure;

FIG. 4 is a block diagram illustrating an example sensor array apparatus, in accordance with embodiments of the present disclosure;

FIGS. 5A, 5B, and 5C are diagrams illustrating example configurations for a sensor array apparatus, in accordance with embodiments of the present disclosure;

FIG. 5D is a diagram illustrating an example wearable article comprising a sensor array apparatus, in accordance with embodiments of the present disclosure;

FIG. 6 is a flow chart illustrating example method for multiple sensor based acoustic respiratory monitoring in accordance with embodiments of the present disclosure;

FIG. 7 is a flow chart illustrating another example method for multiple sensor based acoustic respiratory monitoring in accordance with embodiments of the present disclosure;

FIGS. 8A, 8B, 8C, 8D, and 8E illustrate example user interfaces for a respiratory monitor in accordance with embodiments of the present disclosure;

FIG. 9 is a diagram illustrating an example computing environment in accordance with embodiments of the present disclosure; and

FIG. 10 is a diagram illustrating an example cloud based computing environment in accordance with embodiments of the present disclosure.

### DETAILED DESCRIPTION OF THE INVENTION

In the following detailed description, reference is made to the accompanying drawings that form a part hereof, and in which is shown by way of specific illustrative embodiments in which the embodiments of the technologies described herein may be practiced. These embodiments are described in sufficient detail to enable those skilled in the art to practice the embodiments, and it is to be understood that other embodiments can be utilized and that logical, mechanical and electrical changes can be made without departing from the scope of the present disclosure. The following detailed description is, therefore, not to be taken in a limiting sense. Rather, it is contemplated that the claimed subject matter might also be embodied in other ways, to include different steps or combinations of steps similar to the ones described in this document, in conjunction with other present or future technologies. Moreover, although the terms “step” and “block” may be used herein to connote different elements of methods employed, the terms should not be interpreted as implying any particular order among or between various steps herein disclosed unless and except when the order of individual steps is explicitly described. It should be understood that although the term “physician” is sometimes used herein to refer to one type of user of the described embodiments, a physician is just one example of a healthcare professional that may constitute a user of the disclosed respiratory acoustic monitoring technologies. That is, the embodiments presented herein are not limited to any particular user. For example, it is contemplated that a user may include a patient in some instances.

Accordingly, respiratory acoustic monitoring technologies are disclosed herein. As discussed in greater detail below, in some embodiments, an acoustic respiratory monitoring system includes a sensor array that includes multiple respiratory sounds acquisition sensors (referred to herein individually as acoustic sensor elements), and a respiratory monitoring device that processes acoustic information based on the respiratory sounds produced by a patient's breathing. In some embodiments, the respiratory monitoring device includes a human machine interface (HMI) through which a user, such as a healthcare professional, can select, filter, and/or manipulate signals from one or more acoustic sensor elements of the sensor array, and/or compare aspects of current breathing sound to aspects of previously acquired breathing sounds from the patient. The respiratory monitoring device may also include one or more algorithms that apply logic to correlate aspects of the patient breathing sounds, such as features or patterns, with known adventitious features or patterns for a particular respiratory condition, and present those correlations to the healthcare professional. Example respiratory conditions that may be identified through breathing sound patterns using the embodiments described herein include, but are not limited to, asthma, emphysema, chronic obstructive pulmonary disease (COPD), bronchiectasis, pneumonia, pneumothorax, pneumatocele, other airway diseases, respiratory infections, and other respiratory conditions that impact breathing. The respiratory monitoring device may further record, visualize, or play, respiratory acoustic information acquired from the sensor array in real time. In some implementations, patient breathing sounds are collected by multiple respiratory sounds acquisition sensors contemporaneously. In this way, acoustic respiratory information, as captured from different acoustic sensor elements, may be synchronized in time and processed in different ways as a holistic data set rather than merely as a collection of breathing sounds.

Some embodiments of the respiratory monitor system or device comprise a user interface, such as a graphical user interface provided via a computer display, that includes functionality enabling a healthcare professional to selectively view and/or listen to real-time and/or previously processed breathing sound patterns or other information, and may further include functionality to selectively filter, process, or display acoustic measurement data from one or a portion of the acoustic sensor elements. The user interface may also alert the healthcare professional to areas of the patient's body where disease may be present, such as by displaying on the user interface an indication of a position on the patient corresponding to a detected adventitious feature.

### Overview of Technical Problems, Technical Solutions, and Technological Improvements

Various embodiments of the respiratory acoustic monitoring technologies disclosed herein provide a technological improvement over conventional systems for detecting, monitoring, and/or tracking, acoustic aspects of respiratory conditions. In particular, conventional approaches to respiratory acoustic monitoring is currently limited by the manner in which breathing sounds are sensed, evaluated, and tracked over time. The conventional technologies involve the assessment of a breathing sound captured by a single acoustic sensor (e.g., a stethoscope) for a single breathing cycle (e.g., an inhale and an exhale). For example, a patient may be asked to breathe in and out deeply as a stethoscope is moved to various locations on the patient's chest and back-amplifying the sounds produced by the patient's airway. In other words, for each breathing cycle, only a single data point of information is obtained. Based on a series of these data points a physician is expected to recognize atypical sounds and detect a respiratory condition. Moreover, other than through subjective observations that may be noted by a physician, conventional methods at best provide a snapshot of current symptoms and do not provide for tracking of changes in breathing sounds over time based on direct comparisons of breathing sounds at different time instances.

As discussed in further detail throughout this disclosure, the embodiments of the technologies presented herein capture multiple data points of acoustic respiratory data contemporaneously using multiple acoustic sensor elements distributed about the patient's body, which enables a set of data to be acquired and processed that comprises a diverse set of acoustic data for each inhale-exhale event. Accordingly, a greater context is provided to algorithms used for detecting and classifying adventitious features, such as machine learning models, rules based logic, and/or use of pattern definitions, than by serially captured single-point acoustic data. In this way, these embodiments generate a holistic data set comprising greater context used by algorithms that detect and classify adventitious patterns and track adventitious patterns over time. The utilization of multiple acoustic sensor elements for acoustic respiratory monitoring thus represents a technological improvement in the functionality of the underlying system to detect or predict a patient's condition based on acoustic respiratory data features. Moreover, these embodiments presented herein improve computing resource utilization as a greater quantity of acoustic data may be captured during an examination session in a shorter period of time.

Various anomalies in acoustic respiratory data features (such as the presence of crackles and wheezing, for example) can manifest as a result of the physical deterioration of the structures forming a patient's airway. As such, deep or exaggerated breathing by a patient during an examination may actually create atypical airflows that exacerbate the patient's condition by causing further deterioration. The capture of patient breathing sounds contemporaneously and/or simultaneously by multiple respiratory sounds acquisition sensors distributed about the patient's chest and back reduces the number of times the patient needs to perform such deep breathing cycles to collect a full set of data. Further, to prevent the exacerbation of the patient's condition, the acoustic respiratory monitoring system can generate an alert or other signal indicating when a sufficient data set to perform an analysis is collected, and/or a message to the examining healthcare professional to limit, at least in part, procedures in order to prevent unnecessary exacerbation. For example, in one embodiment, based on evaluating patient breathing sound patterns from the multiple acoustic sensor elements of the sensor array in real time, the acoustic respiratory monitoring system recognizes a feature or pattern associated with a known respiratory condition and recommends that the healthcare professional cease or avoid asking the patient to perform certain breathing actions during examination.

As another aspect, in some embodiments, one or more of the sensor array comprising multiple respiratory sounds acquisition sensors may be arranged on a wearable article, such as a shirt, vest, chest strap, or belt, for example. Such an arrangement of the acoustic sensor elements on a wearable article ensures that each sensor is positioned in approximately the same position across a series of examination sessions so that trends in acoustic respiratory information are more directly comparable. For example, trending information may be computed corresponding to changes in a detected adventitious pattern over a period of time. Arrangement of one or more of the acoustic sensor elements on a wearable article may also facilitate long-term monitoring or ambulatory monitoring of the patient as the acoustic respiratory data may be measured as the patient goes about their daily activities. For example, the wearable article incorporating the sensor array can be used both at a hospital or clinical setting and at the patient's work or home for remote monitoring.

The capture of acoustic respiratory data contemporaneously by multiple acoustic sensor elements distributed about the patient's body enables a set of data to be acquired and processed that comprises a diverse set of acoustic data for each inhale-exhale event. Accordingly, a greater context is provided for detecting and classifying adventitious features, such as by the machine learning models, rules based logic, and/or use of pattern definitions, than serially captured acoustic data from a series of sequential inhale-exhale events using a single sensor. Thus, the utilization of multiple respiratory sounds acquisition sensors for acoustic respiratory monitoring represents a technological improvement in the functionality of the underlying system to detect or predict a patient's condition based on acoustic respiratory data features. Moreover, these embodiments presented herein improve computing resource utilization as a greater number of data points of acoustic data may be captured using the multiple respiratory sounds acquisition sensors for analysis in a shorter period of time.

### Additional Description of the Embodiments

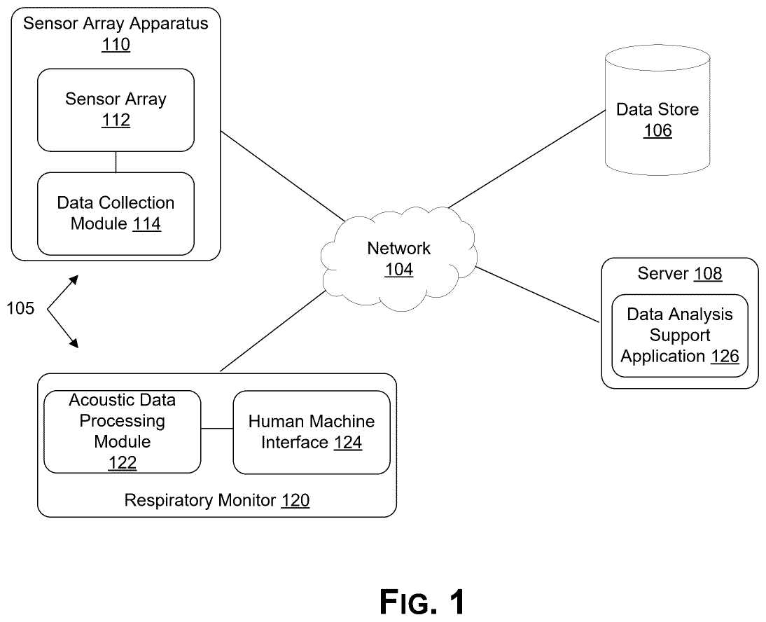

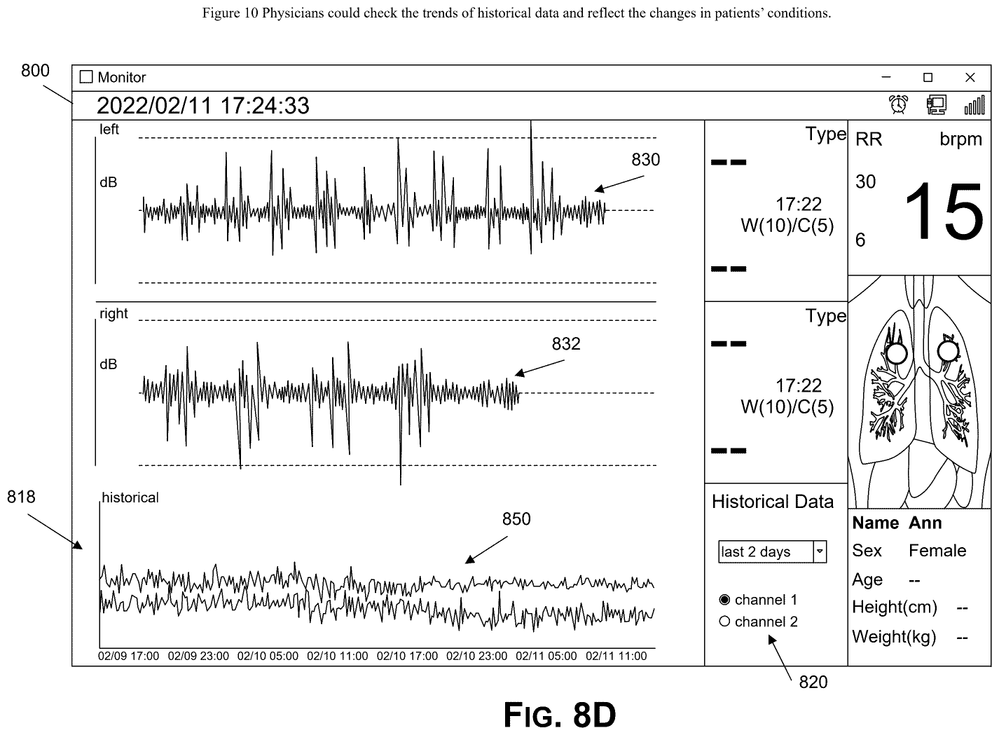

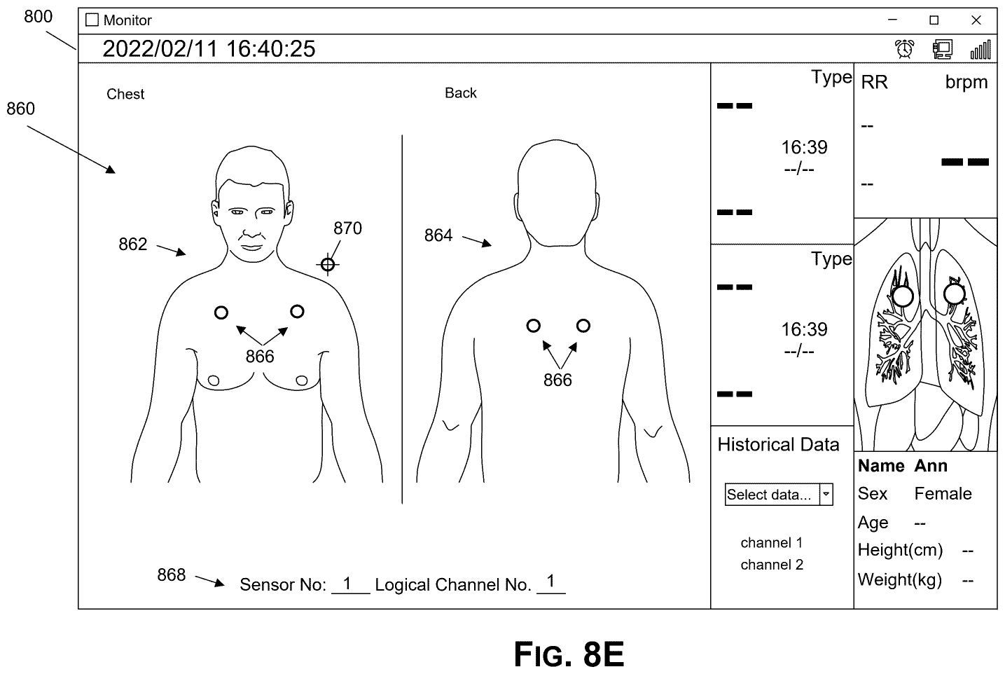

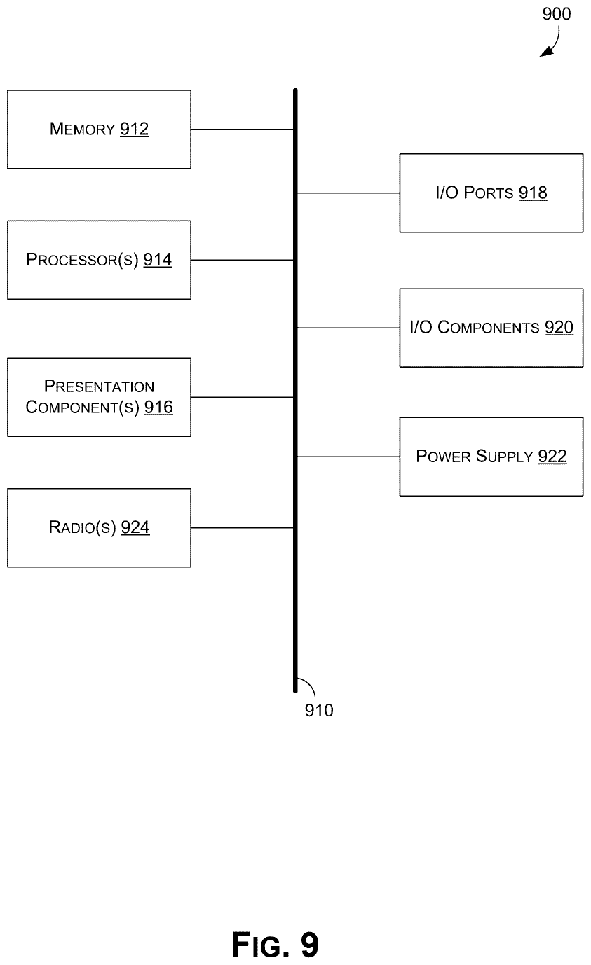

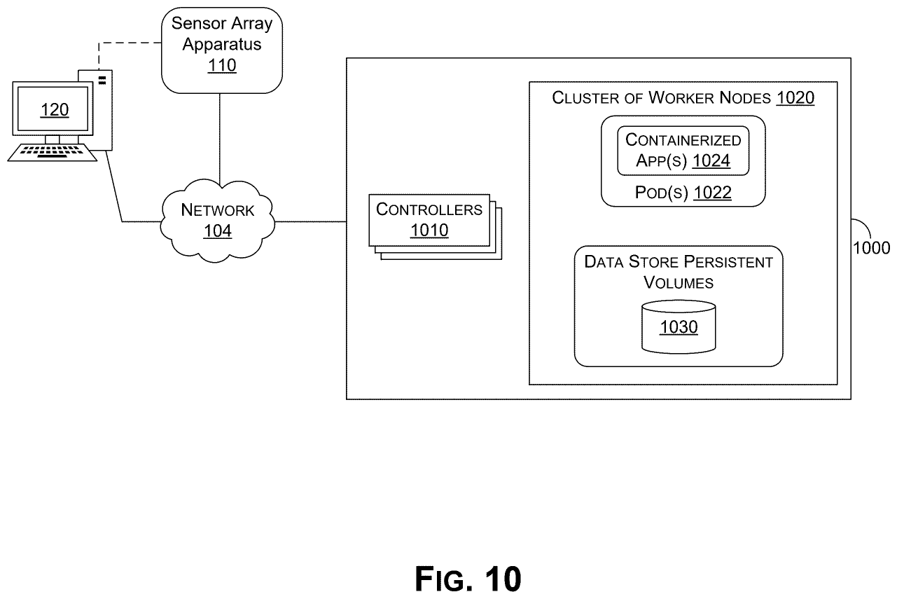

With reference to FIG. 1, FIG. 1 is a diagram of an example operating environment diagram 100 for an acoustic respiratory monitoring system 105 in accordance with some embodiments of the present disclosure. It should be understood that this and other arrangements described herein are set forth only as examples. Other arrangements and elements (e.g., machines, interfaces, functions, orders, groupings of functions, etc.) may be used in addition to or instead of those shown, and some elements may be omitted altogether. Further, many of the elements described in FIG. 1 and/or elsewhere herein are functional entities that may be implemented as discrete or distributed components or in conjunction with other components, and in any suitable combination and location. Various functions described herein as being performed by one or more entities are carried out by hardware, firmware, and/or software. For instance, in some embodiments, some functions are carried out by a processor executing instructions stored in memory as further described with reference to FIG. 9, or within a cloud computing environment as further described with respect to FIG. 10.

Among other components not shown, the operating environment 100 may include an acoustic respiratory monitoring system 105 that comprises a sensor array apparatus 110 and a respiratory monitor 120. Operating environment 100 may also include a network 104, a data store 106, and one or more servers 108. Each of the components shown in FIG. 1 can be implemented, at least in part, via any type of computing device, such as one or more of computing device 900 described in connection to FIG. 9, or within a cloud computing environment 1000 as further described with respect to FIG. 10, for example. These components communicate with each other via network 104, which can be wired, wireless, or both. Network 104 can include multiple networks, or a network of networks, but is shown in simple form so as not to obscure aspects of the present disclosure. By way of example, network 104 can include one or more wide area networks (WANs), one or more local area networks (LANs), one or more public networks, such as the Internet, and/or one or more private networks. Networking environments are commonplace in offices, enterprise-wide computer networks, intranets, and the Internet. Accordingly, network 104 is not described in significant detail.

Respiratory monitor 120 may be implemented as a user device comprising any type of computing device capable of being operated by a user. In some embodiments, the respiratory monitor 120 is a device dedicated to performing respiratory acoustic monitoring functions as described herein. In other embodiments, the respiratory monitor 120 is a multi-purpose device that integrates the respiratory monitoring embodiments described herein with other functionalities. For example, in some implementations, respiratory monitor 120 is embodied as a personal computer (PC), a laptop computer, a mobile device, a smartphone, a tablet computer, a smart watch, a wearable computer, a headset, an augmented reality device, a personal digital assistant (PDA), a handheld communications device, a workstation, any combination of these delineated devices, or any other suitable device.

The sensor array apparatus 110 in FIG. 1 includes a sensor array 112 comprising a plurality of multiple respiratory sounds acquisition sensors (e.g., acoustic sensor elements). In some embodiments, the various acoustic sensor elements of the sensor array each comprise wired or wireless communications functionality to directly communicate collected acoustic measurement data to the respiratory monitor 120 via network 104. In some embodiments, the sensor array apparatus 110 comprises a data collection module 114 and the various acoustic sensor elements of the sensor array 112 are coupled to the data collection module 114. In such embodiments, the data collection module 114 receives the collected acoustic measurement data from the acoustic sensor elements. The data collection module 114 may comprise wired or wireless communications functionality to communicate the collected acoustic measurement data to the respiratory monitor 120 via network 104. Although FIG. 1 illustrates an embodiment where the sensor array apparatus 110 communicates acoustic measurement data via a network 104, in other embodiments the respiratory monitor 120 and the sensor array apparatus 110 are coupled directly together (e.g., either by a direct wireless connection or a wired cable) without using a network. In other words, as further discussed herein in various embodiments, acoustic measurement data from the acoustic sensor elements may be transmitted to the respiratory monitor 120 as analog signals or digital signals, and those signal may be transported from the acoustic sensor elements may be transmitted to the respiratory monitor 120 either through a network (which may be wired, wireless, or a combination thereof) or without using a network (using wired or wireless connections).

As shown in FIG. 1, the respiratory monitor 120 may include an acoustic data processing module 122 and a human machine interface (HMI) 124 (which may, for example, include a graphical user interface (GUI), a user input device, and at least one audio output for listening to acoustic measurement data/breathing sounds). In some embodiments, the acoustic data processing module 122 may be consider as a single application for simplicity, but its functionality can be embodied by one or more applications in practice. The respiratory monitor 120 includes one or more processors and one or more computer-readable media, for executing computer-readable instructions to implement tone or more functions of the respiratory monitor 120 described herein. As further explained below, in some embodiments, the acoustic data processing module 122 operates in conjunction with logic, such as one or more machine learning models, rules based logic, and/or pattern definitions, to evaluate breathing sound patterns received from the sensor array apparatus 110 and perform disease detection and/or classification tasks. These tasks are collectively referred to as disease prediction tasks. In some embodiments, one or more functions attributed herein to the acoustic data processing module 122 are implemented at least in part by a data analysis support application 126 hosted as a server application by the server 108. For example, in one alternate example embodiment, the acoustic data processing module 122 described herein may be implemented in whole or in part by the data analysis support application 126 hosted on the server 108. The sensor array apparatus 110 may send acoustic measurement data to the analysis support application 126 via network 104, and the data analysis support application 126 may send results for display to the HMI 124 via network 104.

In some embodiments, the data store 106 is an element of the acoustic respiratory monitoring system 105. For example, the data store 106 may store historical acoustic measurement data comprising previously collected breathing sound patterns from a patient. The historical acoustic measurement data may be retrieved and used by the acoustic data processing module 122 for trending, or other purposes, as described below. In some embodiments, disease pattern definitions used by the acoustic data processing module 122 to perform disease prediction tasks may be received from the data store 106.

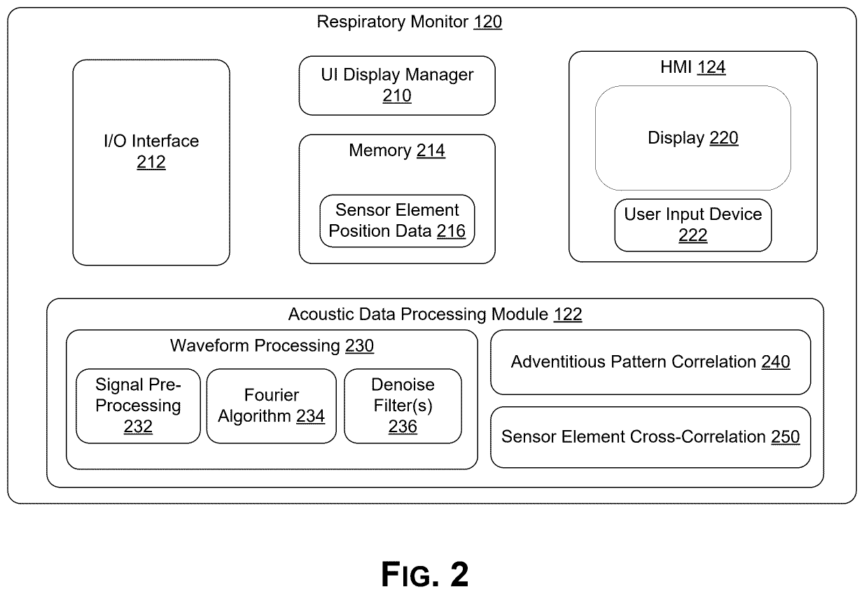

Referring now to FIG. 2, an example respiratory monitor 120 is disclosed. Among other components not shown, the respiratory monitor 120 may include the acoustic data processing module 122 and HMI 124, and also include one or more of a user interface (UI) display manager 210, Input/Output (I/O) interface 212, and a memory 214. In some embodiments, the memory 214 comprises sensor element position data 216 (e.g., information indicating where each of the multiple respiratory sounds acquisition sensors are positioned on the patient under examination). As shown in FIG. 2, the HMI 124 comprises a display 220 to present information and analysis results generated by the acoustic data processing module 122 to a user, such as an examining healthcare professional. In some embodiments, generation and management of user interface screens on the display 220 is controlled by the user interface display manager 210 based on signals from the acoustic data processing module 122 and/or user controls received via HMI 124.

The acoustic data processing module 122 receives the acoustic measurement data collected by the sensor array apparatus 110 via the I/O interface 212. For example, the I/O interface 212 may include a network interface to couple the respiratory monitor 120 to the network 104 via a wired or wireless communication link. In other embodiments, the I/O interface 212 may also, or instead, include an interface to couple the respiratory monitor 120 directly to the sensor array apparatus 110. Wired communication links may comprise a physical medium, such as network cabling, co-axial cables, twisted pair cables, and optical fiber links, or other physical medium. Wireless communication links may be established using wireless technologies such as, but not limited to, an Institute of Electrical and Electronics Engineers (I.E.E.E) standard 802.11 (WiFi), 802.15.4 (Zigbee), industry standard Bluetooth, X-10, or Z-wave, or other wireless protocols.

In some embodiments, such as shown in FIG. 2, the acoustic data processing module 122 includes the functions of waveform processing 230, adventitious pattern correlation 240, and sensor element cross-correlation 250. The waveform processing 230 may include one or more signal pre-processing functions 232. For example, in some embodiments, the pre-processing functions 232 may input the acoustic measurement data collected by the sensor array apparatus 110 and sort the acoustic measurement data into distinct logical channels, where each logical channel generated from the acoustic measurement data carries a stream of acoustic measurement data corresponding to one of the acoustic sensor elements of the sensor array apparatus 110. In some embodiments, the signal pre-processing functions 232 may further execute one or more algorithms to align and/or synchronize the collected acoustic measurement data within each logical channel with respect to time. Acoustic measurement data from a given sensor element, or subset of acoustic sensor elements, may thus be channelized as a discrete logical channel of acoustic measurement data. The pre-processing functions 232 may perform one or more operations to perform a time domain alignment of the acoustic measurement data carried by the different logical channels (for example, based on time stamps). Moreover, the acoustic measurement data may be received from the sensor array apparatus 110 as analog or digitized signals. For embodiments where the acoustic measurement data is received as analog signals, the pre-processing functions 232 may sample the analog signals (e.g., using an analog to digital converter) to generate the logical channels corresponding to a respective sensor element.

As shown in FIG. 2, in some embodiments, waveform processing 230 includes a Fourier algorithm 234 (such as a Fast Fourier transform (FFT) or Discrete Fourier transform (DFT), for example). The Fourier algorithm 234 converts time domain acoustic measurement data into the frequency domain. For example, in some embodiments, the Fourier algorithm 234 receives the plurality of logical channels of acoustic measurement data (e.g., from the signal pre-processing 232) and transform each logical channel of time domain acoustic measurement data into frequency domain spectral components. The Fourier algorithm 234 produces frequency information about the acoustic measurement data which may be used, for example, to compare spectral elements of breathing sound patterns from the patient with spectral elements of breathing sound patterns that are known to correspond to one or more pulmonary/airway diseases.

As further shown in FIG. 2, the waveform processing 230 may further include one or more de-noise filters 236 that filter the acoustic measurement data to reduce environmental noises and/or mitigate extraneous noise signals such as the cardiac noises (e.g., second heart sounds) and human voices. For example, in some embodiments, de-noise filters 236 implements one or more band-pass filters that attenuate spectral components of the acoustic measurement data that do not correspond to breathing sound patterns. In some embodiments, the de-noise filters 236 may apply cross-channel cancelation to attenuate targeted spectral components of the acoustic measurement data. For example, if an extraneous sound (e.g., such as a cardiac related sound) is prominent in one or the logical channels of acoustic measurement data from a particular sensor, then a signal cancelation algorithm may be applied by the de-noise filters 236 to subtract spectral components of that extraneous sound from the acoustic measurement data carried by the other logical channels.

The adventitious pattern correlation 240 receives the acoustic measurement data and evaluates breathing sound patterns captured by the acoustic measurement data, for example, to extract features and classify abnormal respiratory sounds. As further explained below, in some embodiments the adventitious pattern correlation 240 uses one or more of machine learning models, rules based logic, and/or pattern definitions, to evaluate breathing sound patterns received from the sensor array apparatus 110 and perform adventitious pattern detection and/or classification tasks which may be collectively referred to herein as disease prediction tasks. Moreover, in some embodiments, the adventitious pattern correlation 240 includes as input patient acoustic measurement records (e.g., historical acoustic measurement data collected from the patient during previous sessions using the acoustic respiratory monitoring system 105) to perform the disease detection and/or classification tasks. Using the combination of both real-time and historical acoustic measurement data, the adventitious pattern correlation 240 may perform disease prediction tasks that include determinations of predicted diagnoses (e.g., that identify a predicted present disease, illness, or condition) predicted prognoses (e.g. that identify a predicted course of the diagnosed disease, illness, or condition).

The sensor element cross-correlation 250, in some embodiments, cross-correlates adventitious patterns detected by the adventitious pattern correlation 240 back to one or more specific acoustic sensor elements of the sensor array apparatus 110. For example, in some embodiments sensor element positions data 216 (e.g., stored in memory 214) comprises data indicating the position of each sensor element of the sensor array apparatus 110 with respect their location on the patient. When the healthcare professional selects one or more of the acoustic sensor elements (e.g., via user input device 222) in order to view and/or listen to a channel of acoustic measurement data, the sensor element cross-correlation 250 may use the sensor element positions data 216 to identify via the display 220 the acoustic sensor elements producing that acoustic measurement data. Further, in some embodiments, the sensor element cross-correlation 250 may evaluate other logical channels for adventitious patterns based on an adventitious patterns detected on a channel selected by the healthcare professional. For example, when an adventitious pattern is detected and/or classified in one channel, the sensor element cross-correlation 250 may cross-correlate that breathing pattern and/or classification with breathing patterns on other channels. The sensor element cross-correlation 250 may indicate to the healthcare professional (e.g., via the display 220) that an adventitious pattern observable from the currently selected sensor element is either more prominently present, or more clearly defined, in a stream of acoustic measurement data from a different sensor element, and indicate on the display 220 the position of that different sensor element. Conversely, the sensor element cross-correlation 250 may indicate to the healthcare professional other sensor elements where that adventitious pattern appears to be absent, or at least has an amplitude that falls below a threshold level.

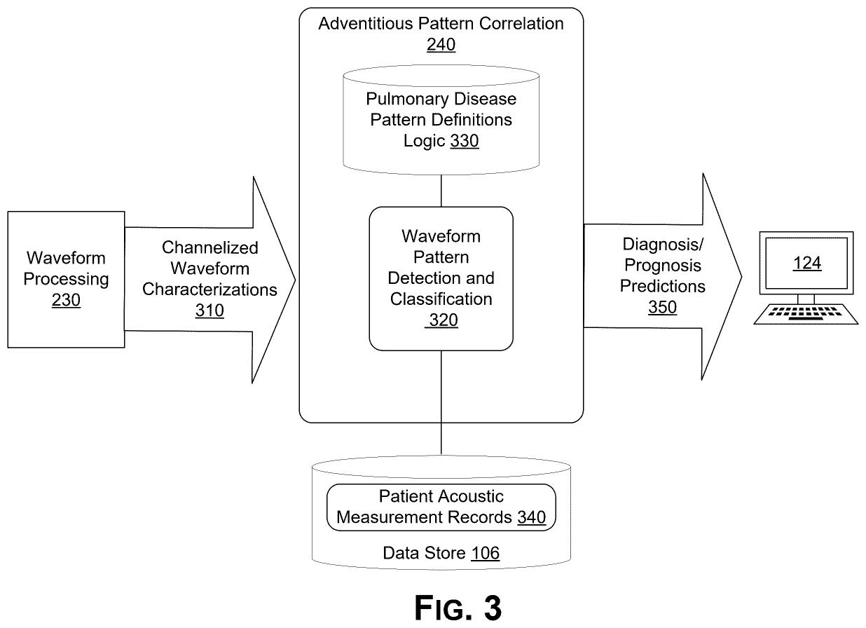

Referring now to FIG. 3, FIG. 3 is a diagram illustrating the adventitious pattern correlation 240 in accordance with embodiments of this disclosure. As illustrated in FIG. 3, the waveform processing 230 of the acoustic data processing module 122 outputs channelized waveform characterizations 310 to the adventitious pattern correlation 240. The channelized waveform characterizations 310 may include the plurality of logical channels of acoustic measurement data. Each logical channel of acoustic measurement data may comprise frequency information (e.g., the frequency domain spectral components computed by the Fourier algorithm 234) generated using the acoustic measurements sensed from the patient by one of the acoustic sensor elements of the sensor array apparatus 110. The adventitious pattern correlation 240 applies the waveform characterizations 310 to waveform pattern detection and classification 320, which evaluates the breathing sound patterns present in the acoustic measurement data to perform disease prediction tasks, such as disease detection and/or classification tasks. These disease prediction tasks may be implemented using pulmonary disease pattern definitions logic 330, which may comprise, for example, machine learning models, rules based logic, a pattern definition database, and/or combinations thereof. For example, pulmonary disease pattern definitions logic 330 may detect the presence of a high-pitched whistling sound occurring while the patient is exhaling—that is characteristic of an adventitious pattern comprising wheezing, or crackling, popping or clicking sounds occurring while the patient is inhaling—that is characteristic of an adventitious pattern comprising crackling. Where the pulmonary disease pattern definitions logic 330 comprises a machine learning model, that machine learning model is not restricted to any particular machine learning model architecture or neural network structure and may comprise, for example and without limitation, a deep neural network, convolutional neural network, or recurrent neural network. The machine learning model may be trained to detect and/or classify adventitious patterns from acoustic measurement data of patient breathing and/or predict a disease, illness, or condition based on the adventitious pattern detected.

In some embodiments, the pulmonary disease pattern definitions logic 330 may be trained and/or programed using ground truth data that includes a combination of acoustic measurement data from patients having known airway diseases and patients known not to have an airway diseases. In some embodiment, the waveform pattern detection and classification 320 may use a pattern matching algorithm or other rules based logic to match the breathing patterns present in the acoustic measurement data to one or more databases of adventitious patterns that correspond to known airway diseases. For example, a waveform signature of the acoustic measurement data may be compared to a plurality of different waveform signatures corresponding to known diseases, illnesses, or conditions to detect and/or classify an adventitious pattern from the acoustic measurement data. Moreover, in some embodiments the channelized waveform characterizations 310 may be evaluated using the pulmonary disease pattern definitions logic 330 as a holistic data set (e.g., a holistic data set of waveform characterization derived from the logical channels) rather than merely considering the acoustic measurement data on an individual logical channel basis.

As another example, sensor element placement information for a sensor element may be paired with acoustic measurement data from that sensor element to produce a paired set of sensor data that is applied to the pulmonary disease pattern definitions logic 330. The paired set of sensor data from each of the plurality of acoustic sensor elements of the sensor array 112 may be evaluated as a whole (considering both breathing sounds and sensor placements) to detect and/or classify the adventitious pattern in the breathing sounds. In some embodiments, the pulmonary disease pattern definitions logic 330 may predict a position on the patient corresponding to detection of the adventitious pattern from multiple acoustic sensor elements, and display that position on the HMI 124. Moreover, the capture of patient breathing sounds contemporaneously by multiple respiratory sounds acquisition sensors distributed about the patient's torso means that the set of data evaluated to detect and/or classify the adventitious patterns comprises a diverse set of acoustic data for each inhale-exhale event, providing greater context for the machine learning models, rules based logic, and/or use of pattern definitions than serially captured acoustic data from a series of sequential inhale-exhale events from a single sensor.

In some embodiments, the waveform pattern detection and classification 320 may further input patient acoustic measurement records 340, which may be used to provide further context to the current set of acoustic measurement data. For example, data from the patient acoustic measurement records 340 may be used to augment the channelized waveform characterizations 310 and applied to the pulmonary disease pattern definitions logic 330 in order to track the progression of a disease or condition over time, and/or predict a prognosis of the course of a disease in addition to a diagnosis.

Because the patient breathing sounds are collected by multiple respiratory sounds acquisition sensors contemporaneously, breathing sound patterns as captured from different acoustic sensor elements may be tracked over time (using the patient acoustic measurement records 340) to determine if a condition is spreading based on changes to what each sensor element measures. For example trending information corresponding to a detected adventitious pattern may be computed using historical acoustic measurement data from the patient acoustic measurement records 340. Trending information may also be computed showing changes in the adventitious pattern as detected over a selected time period based at least in part on the historical acoustic measurement data from patient acoustic measurement records 340.

In some embodiments, the respiratory monitor 120 may track historical data and compute and display trends (e.g., such as short term and/or long term trending lines) indicating changes in a patient's condition. Trending and tracking may be performed on a channel-by-channel basis so that the respiratory monitor 120 may present on HMI 124 breathing sound pattern trends and tracking corresponding to specific acoustic sensor elements selected by the healthcare professional to illustrate if the breathing sound pattern indicates improvements or further deteriorations in one or more certain areas over time. Trending information may include quantitative trending information (e.g., statistics) computed by the acoustic data processing module, in addition to graphical representations. The ability of the respiratory monitor 120 to generate a trend analysis using current and historical acoustic measurement data provides a technical functionality that can facilitate a healthcare professional in prescribing a course of treatment most appropriate to treat the patient's ailment—to a degree that could not be realized by spot-checking breathing sounds using a stethoscope.

The predictions generated by the waveform pattern detection and classification 320 may be output as one or more diagnosis and/or prognosis predictions 350, and displayed onto the HMI 124 as discussed herein, or used for other purposes. In some embodiments, the current set of acoustic measurement data from the sensor array apparatus 110, the channelized waveform characterizations 310 derived from the acoustic measurement data, the one or more diagnosis and/or prognosis predictions 350 produced by the adventitious pattern correlation 240 may be saved to the data store 106 to include in the patient acoustic measurement records 340, for example for use as historical acoustic measurement data with respect to future patient examinations.

Although this disclosure primarily discusses adventitious patterns in breathing in the context of disease, in other embodiments, the respiratory acoustic monitoring described herein may be used for other use cases. For example, the acoustic respiratory monitoring system 105 may also be used to monitor a patient for other respiratory sounds, such as but not limited to rhonchi (gurgling or bubbling sounds during inhalation and/or exhalation caused by fluids), stridor (a noisy or high-pitched breathing sound usually caused by a blockage), cough (a respiratory system reflex usually triggered to clear the airway), and sputum (caused by a presence of thick mucus produced by the lungs). In such embodiments, the pulmonary disease pattern definitions logic 330 used by the waveform pattern detection and classification 320 may include training and/or adventitious pattern definitions corresponding to those conditions.

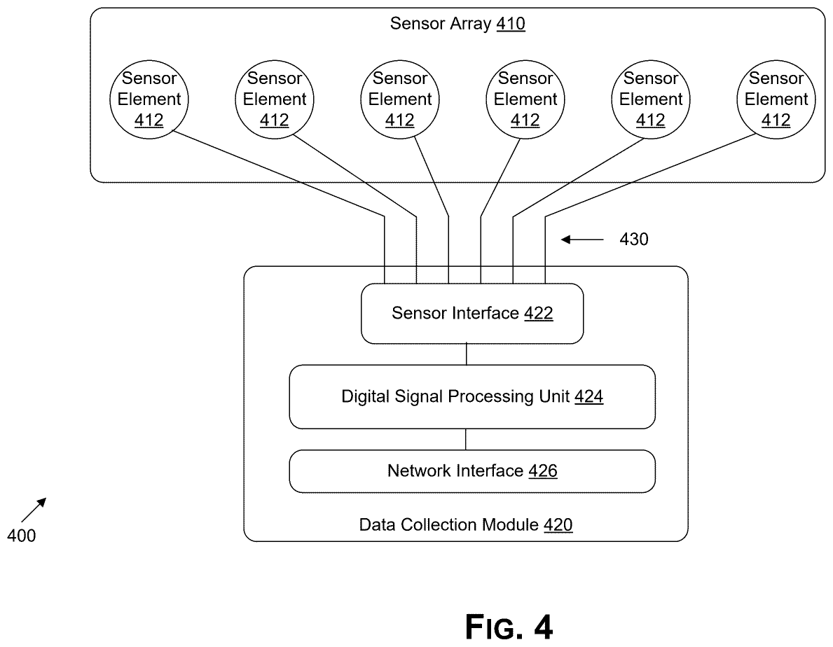

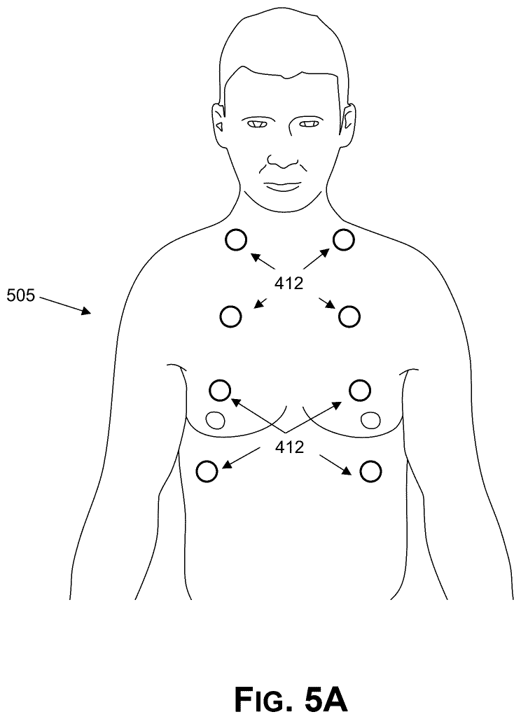

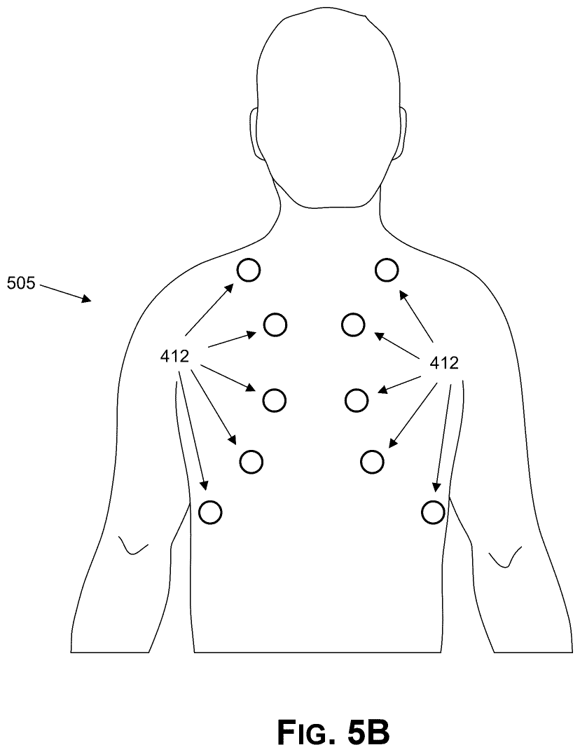

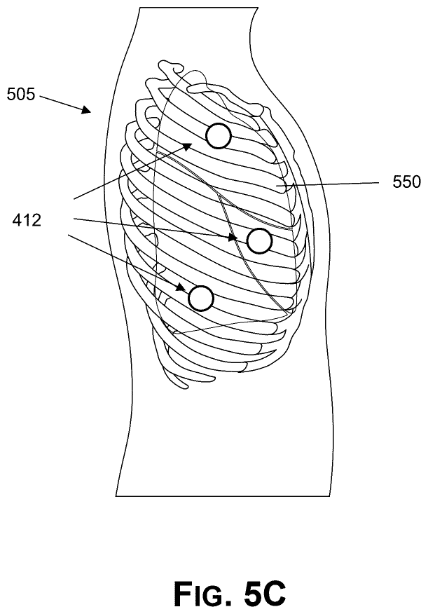

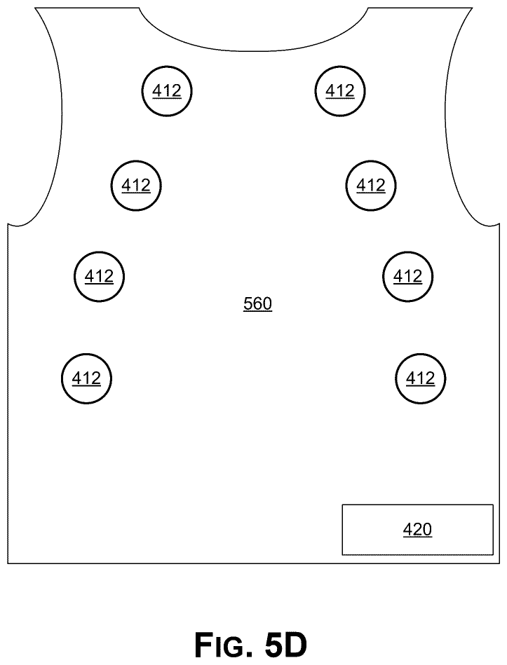

Referring now to FIG. 4 and FIGS. 5A to 5D, one or more examples are illustrated of a sensor array apparatus according to embodiments of this disclosure. Referring to FIG. 4, a sensor array apparatus 400 is illustrated, such as sensor array apparatus 110 discussed above. Sensor array apparatus 400 comprises a sensor array 410 (corresponding to sensor array 112). Sensor array 410 comprises a plurality of respiratory sounds acquisition sensors, shown in FIG. 4 as acoustic sensor elements 412. Acoustic sensor elements 412 may comprise any form of acoustic sensor that detects acoustic signals produced by airflow in the patient's airway during inhalation and exhalation, and converts the acoustic signals into acoustic measurement data that may be carried as signals, such as but not limited to electrical signals over wires or optical signals over optical fiber. Although FIG. 4 illustrates a sensor array 410 comprising six acoustic sensor elements 412, it should be understood that this is for illustrative purposes and that a sensor array 410 may comprise a fewer or greater number of acoustic sensor elements 412.

In some embodiments, one or more of the acoustic sensor elements 412 may be coupled directly to the respiratory monitor, for example using electrical conductors or fiber optics that carry acoustic measurement data to the I/O interface 212. The acoustic sensor elements 412 may comprise wired or wireless network interfaces that transmit acoustic measurement data to the I/O interface 212 via the network 104. In some embodiments, such as shown in FIG. 4, the acoustic measurement data from one or more of the acoustic sensor elements 412 is collected by a data collection module 420. The data collection module 420 receives the acoustic measurement data from the sensor array 410 and communicates that data to the respiratory monitor 120, for example, either through a direct connection to I/O interface 212 or via network 104.

In the embodiment illustrated by FIG. 4, the data collection module 420 may include a sensor interface 422. For example, each of the sensor element 412 may include a corresponding set of connectors 430 (e.g., wires and/or optical fiber) that carry signals with the acoustic measurement data. The sensor interface 422 may comprise one or more ports (such as pluggable ports, for example) that are compatible with receiving the connectors 430 from the acoustic sensor elements 412.

In some embodiments, the data collection module 420 may optionally process the signals from acoustic sensor elements 412 using a digital signal processing unit 424. For example, where the signals from the acoustic sensor elements 412 are analog signals, the digital signal processing component 424 samples the acoustic measurement data to generate digitized acoustic measurement data. In some embodiments, the digital signal processing component 424 may further apply a timestamp to the digitized acoustic measurement data as received from each sensor element 412 to facilitate synchronization of acoustic measurement data by the respiratory monitor 120. The data collection module 420 may further include a network interface 426 which formats the acoustic measurement data for transport via network 104 to the respiratory monitor 120. In some embodiments, the network interface 426 comprises a wireless interface that may communicate the acoustic measurement data to the respiratory monitor 120 using a wireless protocol such as, but not limited to WiFi, Zigbee, Bluetooth, X-10, Z-wave, or other wireless protocols. In other embodiments, the network interface 426 may communicate with the I/O interface 212 via an optical wireless signal.

FIGS. 5A and 5B are diagrams illustrating the positioning of acoustic sensor elements 412 on a patient. To perform a respiratory examination using the acoustic respiratory monitoring system 105, the multiple acoustic sensor elements 412 of the sensor array 410 may be positioned on the patient (e.g., attached against the patient's skin to the chest as shown in FIG. 5A, or back as shown in FIG. 5B,) so that the respiratory monitor 120 receives acoustic measurement data from different positions about the patient's torso 505. As illustrated in FIG. 5C, the acoustic sensor elements 412 may be placed at any location the healthcare professional wants to monitor. In this example of FIG. 5C, the acoustic sensor elements 412 are positioned to capture breathing sounds occurring in specific regions of a patient's internals 550.

The locations where each of the acoustic sensor elements 412 are position on the patient's torso may be entered into the respiratory monitor 120 by the healthcare professional (e.g. via the HMI 124) and stored as the sensor element position data 216 in memory. In some embodiments, the respiratory monitor 120 may read from the patient acoustic measurement records 340 the positions used during prior examinations and display those on the display 220 so that the healthcare professional can again locate the sensor element 412 to the same positions.

In some embodiments, a sensor element 412 may be applied to the patient using a medical adhesive. A sensor element 412 can be either single use (e.g. disposable) or multi-use (e.g. reusable) components. In some embodiments, one or more components of the sensor array apparatus 110 may be integrated into a wearable article such as, but not limited to a shirt, robe, vest, chest strap, or belt. For example, FIG. 5D illustrate a wearable article 560 (in this example, a vest) comprising an arrangement of the acoustic sensor elements 412. Integrating one or more the acoustic sensor elements 420 into a wearable article 560 provides the advantage of ensuring that each of the acoustic sensor elements 412 is positioned in approximately the same position across a series of examination sessions so that trends in breathing sound patterns are more directly comparable. Integration of at least one of the acoustic sensor elements 412 with wearable article 560 may also facilitate long-term monitoring of the patient as the breathing sound patterns may be measured and acoustic monitoring data capture on a more continuous basis as the patient goes about their daily activities. In some embodiments, a sensor array 410 comprises a combination of acoustic sensor elements 412 where one or more of the acoustic sensor elements are applied onto the patient's skin directly, and one or more of the acoustic sensor elements are integrated into a wearable article 560. Such an embodiment permits the respiratory monitor 120 to receive and process acoustic measurement data from one or more predefined standard locations using the acoustic sensor elements of the wearable article, and at the same time from acoustic sensor elements specifically placed at one or more targeted locations of interest or concern to the healthcare professional. Moreover, the wearable article 560 incorporating the sensor array can be used both in hospital setting and settings such as the patient's work or home for remote monitoring. In this example, a data collection module 420 is also integrated into the wearable article 560. In some embodiments, the data collection module 420 may establish a wireless connection with the respiratory monitor 120 (e.g., via network 104) so that the patient wearing the wearable article 560 enjoys a freedom to move about while still being monitored.

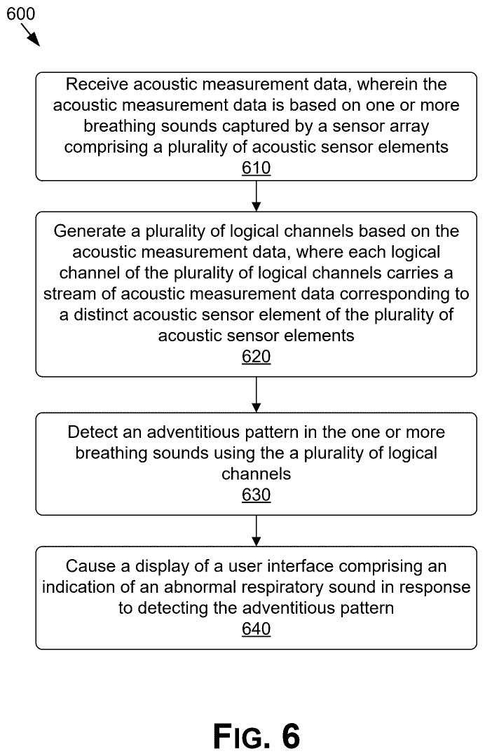

Referring now to FIG. 6, FIG. 6 is a flowchart illustrating a method for multiple sensor based acoustic respiratory monitoring in accordance with embodiments of this disclosure. It should be understood that the features and elements described herein with respect to the method 600 of FIG. 6 can be used in conjunction with, in combination with, or substituted for elements of, any of the other embodiments discussed herein and vice versa. Further, it should be understood that the functions, structures, and other descriptions of elements for embodiments described in FIG. 6 can apply to like or similarly named or described elements across any of the figures and/or embodiments described herein and vice versa. In some embodiments, elements of method 600 are implemented utilizing elements of the acoustic respiratory monitoring system 105 disclosed herein, or other processing device implementing the present disclosure.

The method 600 at 610 includes receiving acoustic measurement data, wherein the acoustic measurement data is based on one or more breathing sounds captured by a sensor array comprising a plurality of acoustic sensor elements. For example, the sensor array may comprises a plurality of respiratory sounds acquisition sensors such as the sensor array 112 of sensor array apparatus 110. The acoustic sensor elements may comprise any form of acoustic sensor that detects acoustic signals produced by airflow in the patient's airway during inhalation and exhalation, and converts the acoustic signals into acoustic measurement data that may be carried as signals, such as but not limited to electrical signals over wires, or optical signals over optical fiber. The plurality of acoustic sensor elements may be distributed, for example across the chest and/or back of a patient, being placed anywhere on the skin the healthcare professional selects. Placement of the acoustic sensor elements may be recorded into the sensor element position data 216 through the HMI 124 as further discussed below. In some embodiments, one or more acoustic sensor elements may be secured to the patient (for example using a medical adhesive), or integrated with a wearable article such as, but not limited to a shirt, robe, vest, chest strap, or belt. While using a wearable article may not facilitate easily relocating acoustic sensor elements, it may assist a patient and/or healthcare professional in more easily placing the acoustic sensor elements in consistent locations over time.

The method 600 at 620 includes generating a plurality of logical channels based on the acoustic measurement data. Each logical channel of the plurality of logical channels may carry a stream of acoustic measurement data corresponding to a distinct acoustic sensor element of the plurality of acoustic sensor elements. That is, the acoustic measurement data collected by the sensor array apparatus may be separately carried and processed as distinct logical channels, where each distinct logical channel carries a stream of acoustic measurement data corresponding to one of the acoustic sensor elements of the sensor array apparatus. Using the logical channels, acoustic measurement data and information derived from that acoustic measurement data, can be correlated back to a specific acoustic sensor element that captured the data for purposes of display and further analysis. In some embodiments, the acoustic data processing module 122 may generate the plurality of logical channels using the acoustic measurement data received from the sensor array apparatus.

The method 600 at 630 includes detecting an adventitious pattern in the one or more breathing sounds using a plurality of logical channels. For example, in some embodiments, breathing sound patterns captured by the acoustic measurement data is evaluated to extract features and classify abnormal respiratory sounds. In some embodiments, detecting the adventitious pattern is performed using one or more of machine learning models, rules based logic, and/or pattern definitions, and may further comprise classification adventitious pattern. Historical acoustic measurement data collected from the patient may be included to perform the adventitious pattern detection and/or classification tasks. The adventitious pattern detection and/or classification tasks may be performed by evaluating each of the plurality of logical channels individually. In other embodiments, adventitious pattern detection and/or classification tasks may be performed based on a holistic data set of waveform characterization derived from the logical channels. In some embodiments, sensor element placement locations may be paired with acoustic measurement data to produce a set of location-measurement data pairs that are evaluated to detect and/or classify the adventitious pattern in the breathing sounds. The use of patient breathing sounds captured contemporaneously by multiple respiratory sounds acquisition sensors distributed about the patient's torso means that the set of data evaluated to detect and/or classify the adventitious patterns comprises a diverse set of acoustic data for each inhale-exhale event. Such a dataset provides greater context for the machine learning models, rules based logic, and/or use of pattern definitions, than serially captured acoustic data from a series of sequential inhale-exhale events from a single sensor. While method 600 may be performed in the context of airway disease, in other embodiments, detecting an adventitious pattern may also comprise detecting and/or classifying other respiratory sounds, such as but not limited to rhonchi (gurgling or bubbling sounds during inhalation and/or exhalation caused by fluids), stridor (a noisy or high-pitched breathing sound usually caused by a blockage), cough (a respiratory system reflex usually triggered to clear the airway), and sputum (caused by a presence of thick mucus produced by the lungs).

In some embodiments, detecting an adventitious pattern may comprise applying the acoustic measurement data to one or more waveform processing algorithms. For example, method 600 may include applying a Fourier algorithm to the plurality of logical channels of acoustic measurement data to transform each channel from time domain acoustic measurement data into frequency domain acoustic measurement data, thereby producing frequency information about the acoustic measurement data which may be used, for example, to compare spectral components of breathing sound patterns from the patient with spectral components of breathing sound patterns that are known to correspond to one or more pulmonary/airway diseases. Other waveform processing may include one or more de-noise filters that filter the acoustic measurement data to reduce environmental noises and/or mitigate extraneous noise signals, such as the cardiac noises (e.g., second heart sounds) and human voices. For example, in some embodiments, a de-noise filters implements one or more band-pass filters that attenuate spectral components of the acoustic measurement data that do not correspond to breathing sound patterns. De-noise filters may apply cross-channel cancelation to attenuate extraneous sound in one logical channel based on acoustic measurement data carried by another logical channel.

The method 600 at 640 includes causing a display of a user interface comprising an indication of an abnormal respiratory sound in response to detecting the adventitious pattern. As further discussed below, indications of adventitious patterns detected in the one or more breathing sounds may be presented on an HMI of a respiratory monitor along with, for example, one or more of graphical representations of the acoustic measurement data, one or more respiratory statistics derived from the acoustic measurement data, and/or trending information computed using historical acoustic measurement data. In some embodiments, the user interface may display abnormal respiratory sounds as represented in selected logical channels, corresponding to acoustic measurement data captured by an associated acoustic sensor element.

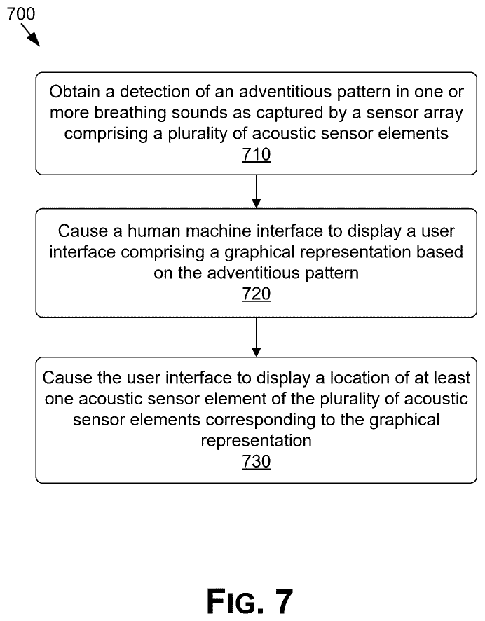

Referring now to FIG. 7, FIG. 7 is a flowchart illustrating a method for multiple sensor based acoustic respiratory monitoring in accordance with embodiments of this disclosure. It should be understood that the features and elements described herein with respect to the method 700 of FIG. 7 can be used in conjunction with, in combination with, or substituted for elements of, any of the other embodiments discussed herein and vice versa. Further, it should be understood that the functions, structures, and other descriptions of elements for embodiments described in FIG. 7 can apply to like or similarly named or described elements across any of the figures and/or embodiments described herein and vice versa. In some embodiments, elements of method 700 are implemented utilizing elements of the acoustic respiratory monitoring system 105 disclosed herein, or other processing device implementing the present disclosure.

The method 700 at 710 includes obtaining a detection of an adventitious pattern in one or more breathing sounds as captured by a sensor array comprising a plurality of acoustic sensor elements. For example, prediction of the adventitious pattern may comprise evaluating one or more streams of acoustic measurement data corresponding to one of the acoustic sensor elements. As described herein, adventitious pattern correlation may be applied to the streams of acoustic measurement data using one or more of machine learning models, rules based logic, and/or pattern definitions, to evaluate breathing sound patterns and perform disease prediction tasks, such as adventitious pattern detection and/or classification, to generate the prediction of the adventitious pattern. The method 700 at 720 includes causing a human machine interface to display a user interface comprising a graphical representation based on the adventitious pattern, and at 730 includes causing the user interface to display a location of at least one acoustic sensor element of the plurality of acoustic sensor elements corresponding to the graphical representation.

For example, in some embodiments, in response to the adventitious pattern detection, the user interface presents the graphical representation of the adventitious pattern and indicates which of the one or more acoustic sensor elements produced the acoustic measurement data that triggered the adventitious pattern detection. When an adventitious pattern is identified on one logical channel, the pattern is cross-correlated with acoustic measurement data on other channels to perform one or more statistics and/or automatically identify other channels where the adventitious pattern is prominent, illustrating the corresponding sensor element positions on the HMI. The method may further output an audio signal (for example, an alert signal and/or an audio signal of the one or more breathing sounds having the adventitious pattern), which may optionally be triggered in response to the adventitious pattern detection.

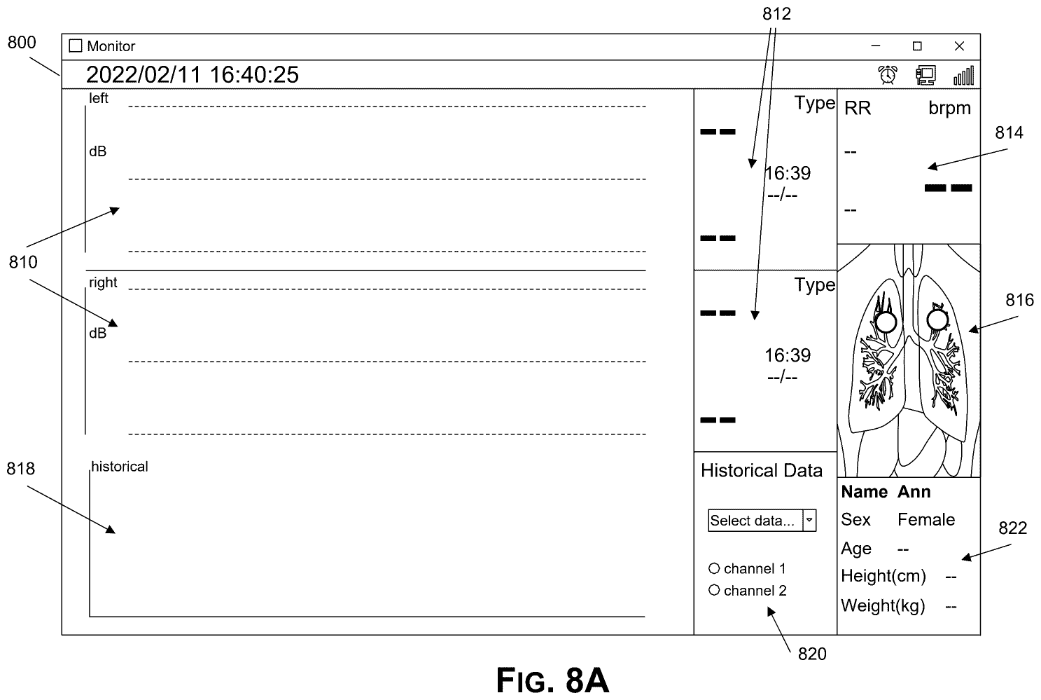

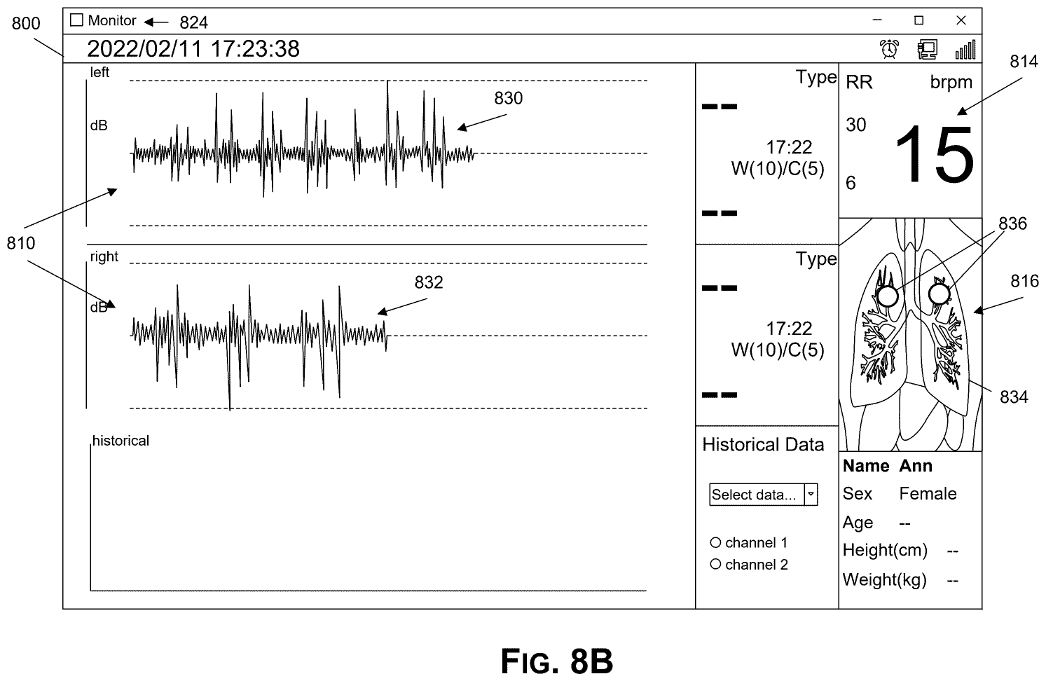

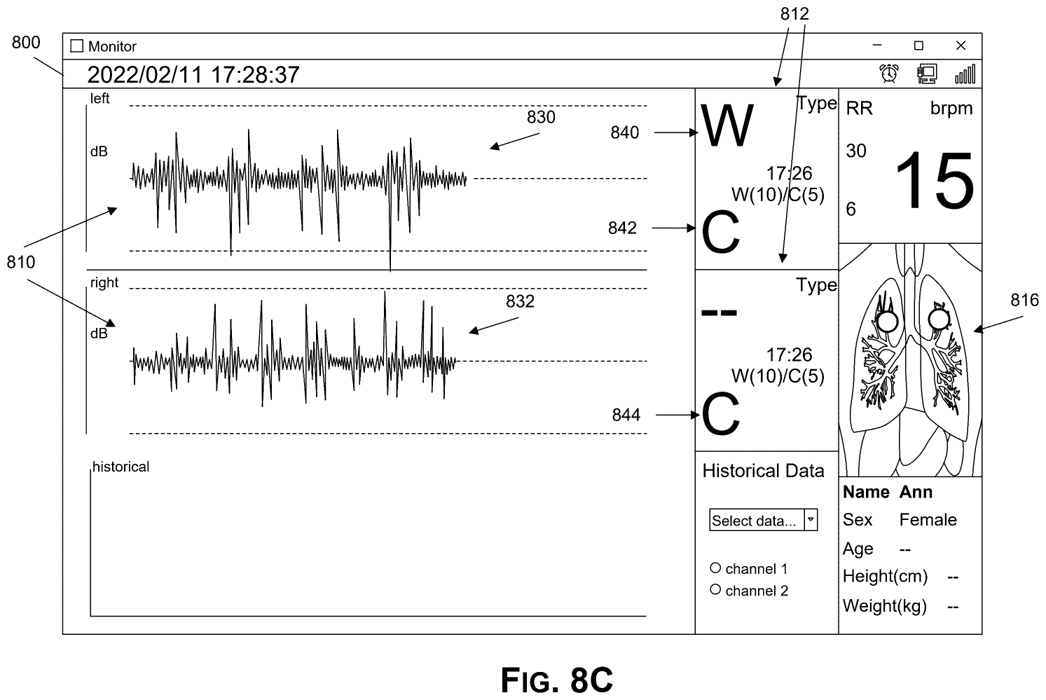

FIGS. 8A-8E illustratively depict aspects of an example user interface 800 generated on an HMI display, such as the display 220 of the HMI 124. In some embodiments, generation and management of the user interface screens shown in FIGS. 8A-8D is controlled by the user interface display manager 210 based on signals from the acoustic data processing module 122 and/or user controls received via user input device 222. For example, user interface display manager 210 may control the presentation of real-time and historic acoustic measurement data, acoustic sensor element locations, diagnosis and prognosis predictions, breathing statistics, or other respiratory data, based on user selections entered into user input device 222 and/or data output by the user interface display manager 210.

Referring to FIG. 8A, the user interface 800 is shown as including a plurality of interface components each presenting different information to the HMI 124. The user interface 800 may include one or more of, but not limited to an interface component 810 comprising a graphical representation of real-time respiratory acoustic measurement data, an interface component 812 presenting indications of detected adventitious patterns in the acoustic measurement data shown in interface component 810, an interface component 814 presenting one or more respiratory statistics, an interface component 816 illustrating a mapping of acoustic sensor element positions, an interface component 818 comprising a graphical representation of historical acoustic measurement data, an interface component 820 comprising user controls for selecting the historical acoustic measurement data presented in interface component 818, and an interface component 822 presenting patient information (e.g., name and/or personal statistics such as age, height and/or weight, for example).

As further illustrated with respect to FIG. 8B, interface component 810 may present a graphical representation of real-time respiratory acoustic measurement data from one or more selected acoustic sensor elements. The interface component 810 may present acoustic measurement data from the one or more logical channels corresponding to the selected acoustic sensor elements of the sensor array 112. In this example, acoustic measurement data from a first logical channel corresponding to a first acoustic sensor element is shown at 830, and acoustic measurement data from a second logical channel corresponding to a second acoustic sensor element is shown at 832. The acoustic measurement data may be presented as a time-domain waveform, or as a frequency-domain spectrograph, based on user selected preferences. In some embodiments, the presence of adventitious pattern components (e.g., as identified by the acoustic data processing module 122) within the presented graphical representation of acoustic measurement data may be highlighted, superimposed, or otherwise indicated in interface component 810. As further illustrated in FIG. 8B, the interface component 814 may present one or more respiratory statistics corresponding to the displayed acoustic measurement data. Respiratory statistics may include, for example, a respiratory rate, or other statistics such as the occurrence time, frequency and/or trending statistics of abnormal respiratory sounds. In some implementations, the respiratory statistics may be computed for a selected window of time (e.g., such as over the prior one minute). In some embodiments, the acoustic data processing module 112 may further include a respiratory sounds detection algorithm to identify normal breathing events, such as inhalation and exhalation states. These acoustic patterns may be converted into breath cycles to calculate a respiratory statistic, such as the respiration rate, that is displayed in interface component 814.

In some embodiments, the acoustic measurement data displayed by interface component 810 may be manually selected via user inputs. The user may use the mapping of acoustic sensor element positions in interface component 816 to select one or more acoustic sensor elements. In this example, the interface component 816 presents an illustration of a patient respiratory system 834 with one or more acoustic sensor element positions 836 indicated with respect to the patient respiratory system 834. In some embodiments, the acoustic sensor element positions 836 may be determined from sensor element position data 216 previously entered into memory 214. The user may interact with the interface component 816 (e.g., by moving a pointer via user input device 222) to select which of the presented acoustic sensor elements to present in interface component 810. For example, the user may select an acoustic sensor placed on the patient's chest to observe real-time respiratory sounds from patient's bronchi. The user interface display manager 210 may respond to the selection by displaying acoustic measurement data from the logical channel corresponding to the selected acoustic sensor element(s). In some embodiments, the user interface 800 may further include a monitor control 824, which when selected causes the HMI 124 to output audio of the breathing sounds corresponding to the displayed acoustic measurement data. In some embodiments, the acoustic sensor elements may be automatically selected for display by the respiratory monitor 120 based on the detection of adventitious patterns. For example, in some embodiments, a user may select a filter to control the user interface 800 to automatically display one or more logical channels of acoustic measurement data where a specified adventitious pattern is detected (such as wheezing or crackling, for example).

Referring to FIG. 8C, the user interface 800 may include an interface component 812 that displays indications of detected adventitious patterns in the acoustic measurement data shown in interface component 810. The indications may be based on adventitious patterns detected and/or classified by the acoustic data processing module 122. For example, here an adventitious pattern indicator 840 displays a “W” to indicate that an adventitious pattern classified as wheezing has been detected in the first logical channel of acoustic measurement data shown at 830, and adventitious pattern indicator 842 displays a “C” to indicate that an adventitious pattern classified as crackling has also been detected in the first logical channel of acoustic measurement data shown at 830. Also, an adventitious pattern indicator 844 displays a “C” to indicate that an adventitious pattern classified as crackling has been detected in the second logical channel of acoustic measurement data shown at 832. Other pattern indicators may be use to indicate a classification of other detected adventitious patterns. The indications of detected adventitious patterns may correspond to the diagnosis and/or prognosis predictions 350 generated by the adventitious pattern correlation 240 and used by the healthcare professional to support clinical decision. In some embodiments, the user interface 800 may further display an indication of a position on a patient that corresponds to the adventitious pattern indicated by an adventitious pattern indicator 840 (e.g., by highlighting the location of a sensor in interface component 816).

Referring to FIG. 8D, the user interface 800 may include interface component 818 comprising a graphical representation of historical acoustic measurement data as shown at 850, and interface component 820 comprising user controls for selecting the historical acoustic measurement data presented in interface component 818. In some embodiments, the historical acoustic measurement data 850 is obtained from the patient acoustic measurement records 340. Healthcare professionals reviewing the historical acoustic measurement data 850 may compare that data to the channels of displayed real time acoustic measurement data, such as shown at 830 and 832, for example. The historical acoustic measurement data 850 supports the healthcare professional's tracking of historical data of abnormal respiratory sounds and may include graphical representations of trend lines or a display of other trending statistics in the interface component 818, to indicate changes that have occurred in a patient's conditions. The healthcare professional may select the scope of historical acoustic measurement data and/or trending statistics using the user controls selected in interface component 820 (e.g., based on a selected time period and/or prior duration of time such as, “today,” “last week,” “last month,” “last 2 days,” “last 3 days,” “last 7 days,” or other time period). The healthcare professional may also select between different available channels of historical acoustic measurement data to display in the interface component 818.

Referring to FIG. 8E, the user interface 800 may include interface component 860 for specifying the placement of the acoustic sensor elements with respect to the patient's torso. As previously discussed, the acoustic sensor elements may be placed at any location the healthcare professional wants to monitor to capture breathing sounds occurring in specific regions of a patient's airway and/or lungs. In some embodiments, the user interface 800 presents a representation of a patient's chest (at 862) and a display of a patient's back (at 864). The healthcare professional may use the chest and back representations to indicate where acoustic sensor elements are positioned on the patient. For example, in the embodiment shown in FIG. 8E, two representations of acoustic sensor elements 866 are shown as placed on the patient's chest, and two representations of acoustic sensor elements 866 are shown as placed on the patient's back. In some embodiments, the user interface 800 may include a pointer 870 that the healthcare professional can move within the interface component 840 (e.g., using user input device 222) to specify where a sensor element is positioned, and the user interface 800 will place a representation of a sensor element 850 at that location. The placement of acoustic sensor elements as entered into interface component 840 may be saved to the sensor element position data 216. In some embodiments, as the healthcare professional specifies sensor element positions, the interface component 840 may include an input field 868 to specify the logical channel assigned to the corresponding sensor element. In other embodiments, the acoustic data processing module 122 may automatically assign logic channels corresponding to each sensor element entered via the interface component 840.

Accordingly, we have described various aspects of improved technologies for monitoring and detecting respiratory conditions. It is understood that various features, sub-combinations, and modifications of the embodiments described herein are of utility and may be employed in other embodiments without reference to other features or sub-combinations. Moreover, the order and sequences of steps shown in the example methods 600 and 700 are not meant to limit the scope of the present disclosure in any way and, in fact, the steps may occur in a variety of different sequences within embodiments hereof. Such variations and combinations thereof are also contemplated to be within the scope of embodiments of this disclosure.

### Other Example Embodiments

In some embodiments, a computerized system for acoustic respiratory monitoring system is provided such as described in any of the embodiments above. As an example, a system comprises one or more computer processors and computer memory having computer executable instructions embodied thereon, that, when executed by the one or more processors perform operations. The operations comprise receiving acoustic measurement data derived from one or more breathing sounds captured by a sensor array comprising a plurality of acoustic sensor elements. The operations also comprise generating a plurality of logical channels based on the acoustic measurement data. The operations further comprise detecting an adventitious feature in the one or more breathing sounds using the plurality of logical channels. The operations further comprise causing a display, via a user interface, of an indication of an abnormal respiratory sound in response to detecting the adventitious feature.

Advantageously, and as discussed in further detail throughout this disclosure, this and one or more other embodiments presented herein capture multiple data points of acoustic respiratory data contemporaneously using multiple acoustic sensor elements distributed about the patient's body, which enables a set of data to be acquired and processed that comprises a diverse set of acoustic data for each inhale-exhale event. These and other embodiments improve exiting computing technologies by providing new or improved functionality to respiratory monitoring applications as a greater context is provided to algorithms used for detecting and classifying adventitious features, such as machine learning models, rules based logic, and/or use of pattern definitions, than by serially captured single-point acoustic data. In this way, these embodiments generate a holistic data set comprising greater context used by algorithms that detect and classify adventitious patterns and track adventitious patterns over time. The utilization of multiple acoustic sensor elements for acoustic respiratory monitoring thus represents a technological improvement in the functionality of the underlying system to detect or predict a patient's condition based on acoustic respiratory data features. Moreover, these embodiments presented herein improve computing resource utilization as a greater quantity of acoustic data may be captured during an examination session in a shorter period of time.

In any combination of the above embodiments of the system, each logical channel of the plurality of logical channels carries a stream of acoustic measurement data corresponding to a distinct acoustic sensor element of the plurality of acoustic sensor elements.

In any combination of the above embodiments of the system, the operations further comprise receiving the acoustic measurement data from the sensor array via a network.

In any combination of the above embodiments of the system, the operations further comprise processing the acoustic measurement data using at least one of: a Fourier algorithm to transform a stream of acoustic measurement data from each logical channel from time domain acoustic measurement data into frequency domain acoustic measurement data; and a de-noise filter to attenuate spectral components of the acoustic measurement data that do not correspond to breathing sound features.

In any combination of the above embodiments of the system, the operations further comprise causing the user interface to display a location of at least one of the plurality of acoustic sensor elements corresponding to the adventitious feature.

In any combination of the above embodiments of the system, the operations further comprise causing the user interface to display a stream of acoustic measurement data corresponding to a first acoustic sensor element of the plurality of acoustic sensor elements in response to a user input selection of the first acoustic sensor element.

In any combination of the above embodiments of the system, each logical channel of the plurality of logical channels carries a stream of acoustic measurement data corresponding to a distinct acoustic sensor element of the plurality of acoustic sensor elements, the operations further comprising: causing the user interface to display a representation of a first stream of acoustic measurement data from a first logical channel of the plurality of logical channels in response to user input selecting a first acoustic sensor element associated with the first logical channel.

In any combination of the above embodiments of the system, the operations further comprise causing the user interface to display historical acoustic measurement data obtained from a patient acoustic measurement record.

In any combination of the above embodiments of the system, the operations further comprise obtaining historical acoustic measurement data from a patient acoustic measurement record; computing trending information corresponding to changes in the adventitious feature as detected over a selected time period based at least in part on the historical acoustic measurement data; and causing the user interface to display the trending information.

In any combination of the above embodiments of the system, the indication of the abnormal respiratory sound includes a classification of the adventitious feature.

In any combination of the above embodiments of the system, each logical channel of the plurality of logical channels carries a stream of acoustic measurement data corresponding to a distinct acoustic sensor element of the plurality of acoustic sensor elements, the operations further comprising: detecting the adventitious feature based on applying one or more of the plurality of logical channels to a disease pattern definitions logic.

In any combination of the above embodiments of the system, the system further comprises a sensor array apparatus comprising a wearable article, wherein the plurality of acoustic sensor elements are incorporated with the wearable article.

In any combination of the above embodiments of the system, the operations further comprise causing the user interface to display an indication of a position on a patient corresponding to detection of the adventitious feature.

As another example embodiment, a method for multiple sensor based acoustic respiratory monitoring is provided. The method comprises receiving acoustic measurement data derived from one or more breathing sounds as captured by a sensor array comprising a plurality of acoustic sensor elements. The method further comprises generating a plurality of logical channels based on the acoustic measurement data. The method further comprises detecting an adventitious feature in the one or more breathing sounds using the plurality of logical channels. The method further comprises causing a display of a user interface comprising an indication of an abnormal respiratory sound in response to detecting the adventitious feature.

Advantageously, and as discussed in further detail throughout this disclosure, this and one or more other embodiments presented herein capture multiple data points of acoustic respiratory data contemporaneously using multiple acoustic sensor elements distributed about the patient's body, which enables a set of data to be acquired and processed that comprises a diverse set of acoustic data for each inhale-exhale event. These and other embodiments improve exiting computing technologies by providing new or improved functionality to respiratory monitoring applications as a greater context is provided to algorithms used for detecting and classifying adventitious features, such as machine learning models, rules based logic, and/or use of pattern definitions, than by serially captured single-point acoustic data. In this way, these embodiments generate a holistic data set comprising greater context used by algorithms that detect and classify adventitious patterns and track adventitious patterns over time. The utilization of multiple acoustic sensor elements for acoustic respiratory monitoring thus represents a technological improvement in the functionality of the underlying system to detect or predict a patient's condition based on acoustic respiratory data features. Moreover, these embodiments presented herein improve computing resource utilization as a greater quantity of acoustic data may be captured during an examination session in a shorter period of time.

In any combination of the above embodiments of the method, each logical channel of the plurality of logical channels carries a stream of acoustic measurement data corresponding to a distinct acoustic sensor element of the plurality of acoustic sensor elements.

In any combination of the above embodiments of the method, the method further comprises causing the user interface to display a location of at least one of the plurality of acoustic sensor elements corresponding to the adventitious feature.

In any combination of the above embodiments of the method, the method further comprises causing the user interface to display trending information computed at least in part from historical acoustic measurement data obtained from a patient acoustic measurement record.

In any combination of the above embodiments of the method, the method further comprises determining a classification of the adventitious feature based on applying one or more of the plurality of logical channels to a disease pattern definitions logic; and wherein the indication of the abnormal respiratory sound includes the classification of the adventitious feature.

As another example embodiment, an acoustic respiratory monitoring system, is presented. The system comprises a sensor array apparatus comprising a sensor array that includes a plurality of acoustic sensor elements, and one or more processors coupled to a memory. The one or more processors to perform acoustic data processing operations. The operations comprise processing a plurality of logical channels that carry acoustic measurement data derived from one or more breathing sounds as captured by the plurality of acoustic sensor elements. Each logical channel of the plurality of logical channels carries a stream of acoustic measurement data corresponding to an acoustic sensor element of the plurality of acoustic sensor elements. The operations further comprise detecting an adventitious feature in the one or more breathing sounds using the stream of acoustic measurement data from one or more logical channels of the plurality of logical channels, and causing a human machine interface to display an indication of an abnormal respiratory sound in response to detecting the adventitious feature.

Advantageously, and as discussed in further detail throughout this disclosure, this and one or more other embodiments presented herein capture multiple data points of acoustic respiratory data contemporaneously using multiple acoustic sensor elements distributed about the patient's body, which enables a set of data to be acquired and processed that comprises a diverse set of acoustic data for each inhale-exhale event. These and other embodiments improve exiting computing technologies by providing new or improved functionality to respiratory monitoring applications as a greater context is provided to algorithms used for detecting and classifying adventitious features, such as machine learning models, rules based logic, and/or use of pattern definitions, than by serially captured single-point acoustic data. In this way, these embodiments generate a holistic data set comprising greater context used by algorithms that detect and classify adventitious patterns and track adventitious patterns over time. The utilization of multiple acoustic sensor elements for acoustic respiratory monitoring thus represents a technological improvement in the functionality of the underlying system to detect or predict a patient's condition based on acoustic respiratory data features. Moreover, these embodiments presented herein improve computing resource utilization as a greater quantity of acoustic data may be captured during an examination session in a shorter period of time.

In any combination of the above embodiments of the system, the sensor array apparatus further comprises a wearable article, wherein the plurality of acoustic sensor elements are incorporated with the wearable article.

### Example Computing Environments

Having described various implementations, several example computing environments suitable for implementing embodiments of the disclosure are now described, including an example computing device and an example distributed computing environment in FIGS. 9 and 10, respectively. With regard to FIG. 9, one example operating environment for implementing aspects of the technology described herein is shown and designated generally as computing device 900. For example, in some embodiments, one or more aspects of the respiratory monitor 120 are implemented using computing device 900. Computing device 900 is just one example of a suitable computing environment and is not intended to suggest any limitation as to the scope of use or functionality of the technology described herein. Neither should the computing device 900 be interpreted as having any dependency or requirement relating to any one or combination of components illustrated.

The technology described herein can be described in the general context of computer code or machine-usable instructions, including computer-executable instructions such as program components, being executed by a computer or other machine, such as a personal data assistant or other handheld device. Generally, program components, including routines, programs, objects, components, data structures, and the like, refer to code that performs particular tasks or implements particular abstract data types. Aspects of the technology described herein can be practiced in a variety of system configurations, including handheld devices, consumer electronics, general-purpose computers, and specialty computing devices. Aspects of the technology described herein can also be practiced in distributed computing environments where tasks are performed by remote-processing devices that are linked through a communications network.

With continued reference to FIG. 9, computing device 900 includes a bus 910 that directly or indirectly couples the following devices: memory 912, one or more processors 914, one or more presentation components 916, input/output (I/O) ports 918, I/O components 920, an illustrative power supply 922, and a radio(s) 924. Bus 910 represents one or more busses (such as an address bus, data bus, or combination thereof). Although the various blocks of FIG. 9 are shown with lines for the sake of clarity, it should be understood that one or more of the functions of the components can be distributed between components. For example, a presentation component 916, such as a display device, can also be considered an I/O component 920. The diagram of FIG. 9 is merely illustrative of an example computing device that can be used in connection with one or more aspects of the technology described herein. Distinction is not made between such categories as “workstation,” “server,” “laptop,” “tablet,” “smart phone,” or “handheld device,” as all are contemplated within the scope of FIG. 9 and refer to “computer” or “computing device.”

Memory 912 is a non-transient computer storage media in the form of volatile and/or nonvolatile memory. The memory 912 can be removable, non-removable, or a combination thereof. Example memory 912 includes solid-state memory, hard drives, flash drives, and/or optical-disc drives. Computing device 900 includes one or more processors 914 that read data from various entities, such as bus 910, memory 912, or I/O components 920. In some embodiments, acoustic data processing module 122 and/or other operations of the respiratory monitor 120 are implemented at least in part by the processors 914.

Presentation component(s) 916 present data indications to a user or other device and in some embodiments comprises the HMI 124 used by respiratory monitor 120 to present acoustic measurement data as textual, graphical, and/or audio outputs, as described herein. Example presentation components 916 include a display device, speaker, printing component, and vibrating component. I/O port(s) 918 allow computing device 900 to be logically coupled to other devices, including I/O components 920, some of which can be built in.

Illustrative I/O components 920 include a microphone, joystick, game pad, satellite dish, scanner, printer, display device, wireless device, a controller (such as a keyboard, and a mouse), a natural user interface (NUI) (such as touch interaction, pen (or stylus) gesture, and gaze detection), and the like. In aspects, a pen digitizer (not shown) and accompanying input instrument (also not shown but which can include, by way of example only, a pen or a stylus) are provided in order to digitally capture freehand user input. The connection between the pen digitizer and processor(s) 914 can be direct or via a coupling utilizing a serial port, parallel port, and/or other interface and/or system bus known in the art. Furthermore, the digitizer input component can be a component separated from an output component, such as a display device, or in some aspects, the usable input area of a digitizer can be coextensive with the display area of a display device, integrated with the display device, or can exist as a separate device overlaying or otherwise appended to a display device. Any and all such variations, and any combination thereof, are contemplated to be within the scope of aspects of the technology described herein.

Some embodiments of example computing device 900 may include a neural network inference engine (not shown). A neural network inference engine comprises a neural network coprocessor, such as a graphics processing unit (GPU), configured to execute a deep neural network (DNN) and/or machine learning models. In some embodiments, functions such as the waveform pattern detection and classification 320, pulmonary disease pattern definitions logic 330, or other operations of the adventitious pattern correlation 240 and/or acoustic data processing module 122 may be executed at least in part using a neural network inference engine.

The computing device 900, in some embodiments, is equipped with depth cameras, such as stereoscopic camera systems, infrared camera systems, RGB camera systems, or combinations of these, for gesture detection and recognition. Additionally, the computing device 900, in some embodiments, is equipped with accelerometers or gyroscopes that enable detection of motion. The output of the accelerometers or gyroscopes can be provided to the display of the computing device 900 to render immersive augmented reality or virtual reality. A computing device, in some embodiments, includes radio(s) 924. The radio 924 transmits and receives radio communications. The computing device can be a wireless terminal adapted to receive communications and media over various wireless networks. For example, in some embodiments the I/O interface 212 comprises a wireless network interface that includes one or more of radios 924.

FIG. 10 is a diagram illustrating a distributed or cloud based computing environment 1000 for implementing one or more aspects of the acoustic data processing module 122 discussed with respect to any of the embodiments discussed herein. Cloud based computing environment 1000 comprises one or more controllers 1010 that each comprises one or more processors and memory, each programmed to execute code to implement at least part of the acoustic data processing module 122. In one embodiment, the one or more controllers 1010 comprise server components of a data center. The controllers 1010 may be configured to establish a cloud based computing platform executing aspects of the acoustic data processing module 122. For example, in some embodiments, one or more operations of the acoustic data processing module 122 and/or data analysis support application 126 are virtualized network services running on a cluster of worker nodes 1020 established on the controllers 1010. For example, the cluster of worker nodes 1020 can include one or more of Kubernetes (K8s) pods 1022 orchestrated onto the worker nodes 1020 to realize one or more containerized applications 1024 to implement the acoustic data processing module 122 and/or data analysis support application 126. In some embodiments, the respiratory monitor 120, sensor array apparatus 110, and/or HMI 124, can be coupled to the controllers 1010 by network 104 (for example, a public network such as the Internet, a proprietary network, or a combination thereof). In such an embodiment, one or both of the acoustic data processing module 122 and/or data analysis support application 126 are at least partially implemented by the containerized applications 1024. In some embodiments, the cluster of worker nodes 1020 includes one or more data store persistent volumes 1030 that implement the data store 106.

In various alternative embodiments, system and/or device elements, method steps, or example implementations described throughout this disclosure can be implemented at least in part using one or more computer systems, field programmable gate arrays (FPGAs), application specific integrated circuits (ASICs), or similar devices comprising a processor coupled to a memory and executing code to realize that elements, processes, or examples, said code stored on a non-transient hardware data storage device. Therefore, other embodiments of the present disclosure can include elements comprising program instructions resident on computer readable media which, when implemented by such computer systems, enable them to implement the embodiments described herein. As used herein, the terms “computer readable media” and “computer storage media” refer to tangible memory storage devices having non-transient physical forms and includes both volatile and nonvolatile, removable and non-removable media. Such non-transient physical forms can include computer memory devices, such as but not limited to: punch cards, magnetic disk or tape, or other magnetic storage devices, any optical data storage system, flash read only memory (ROM), non-volatile ROM, programmable ROM (PROM), erasable-programmable ROM (E-PROM), Electrically erasable programmable ROM (EEPROM), random access memory (RAM), CD-ROM, digital versatile disks (DVD), or any other form of permanent, semi-permanent, or temporary memory storage system of device having a physical, tangible form. By way of example, and not limitation, computer-readable media can comprise computer storage media and communication media. Computer storage media does not comprise a propagated data signal. Program instructions include, but are not limited to, computer executable instructions executed by computer system processors and hardware description languages, such as Very High Speed Integrated Circuit (VHSIC) Hardware Description Language (VHDL).

Many different arrangements of the various components depicted, as well as components not shown, are possible without departing from the scope of the claims below. Embodiments in this disclosure are described with the intent to be illustrative rather than restrictive. Alternative embodiments will become apparent to readers of this disclosure after and because of reading it. Alternative means of implementing the aforementioned can be completed without departing from the scope of the claims below. Certain features and sub-combinations are of utility and can be employed without reference to other features and sub-combinations and are contemplated within the scope of the claims.

1. A system comprising: one or more computer processors; computer memory having computer executable instructions embodied thereon, that, when executed by the one or more processors perform operations comprising: receiving acoustic measurement data derived from one or more breathing sounds captured by a sensor array comprising a plurality of acoustic sensor elements; generating a plurality of logical channels based on the acoustic measurement data; detecting an adventitious feature in the one or more breathing sounds using the plurality of logical channels; and causing a display, via a user interface, of an indication of an abnormal respiratory sound in response to detecting the adventitious feature.

2. The system of claim 1, the operations further comprising: receiving the acoustic measurement data from the sensor array via a network.

3. The system of claim 1, the operations further comprising processing the acoustic measurement data using at least one of: a Fourier algorithm to transform a stream of acoustic measurement data from each logical channel from time domain acoustic measurement data into frequency domain acoustic measurement data; and a de-noise filter to attenuate spectral components of the acoustic measurement data that do not correspond to breathing sound features.

4. The system of claim 1, wherein each logical channel of the plurality of logical channels carries a stream of acoustic measurement data corresponding to a distinct acoustic sensor element of the plurality of acoustic sensor elements, the operations further comprising at least one of: causing the user interface to display a location of at least one of the plurality of acoustic sensor elements corresponding to the adventitious feature; causing the user interface to display a stream of acoustic measurement data corresponding to a first acoustic sensor element of the plurality of acoustic sensor elements in response to a user input selection of the first acoustic sensor element; and causing the user interface to display a representation of a first stream of acoustic measurement data from a first logical channel of the plurality of logical channels in response to user input selecting a first acoustic sensor element associated with the first logical channel.

5. The system of claim 1, the operations further comprising: causing the user interface to display historical acoustic measurement data obtained from a patient acoustic measurement record.

6. The system of claim 1, the operations further comprising: obtaining historical acoustic measurement data from a patient acoustic measurement record; computing trending information corresponding to changes in the adventitious feature as detected over a selected time period based at least in part on the historical acoustic measurement data; and
causing the user interface to display the trending information.

7. The system of claim 1, wherein the indication of the abnormal respiratory sound includes a classification of the adventitious feature.

8. The system of claim 1, wherein each logical channel of the plurality of logical channels carries a stream of acoustic measurement data corresponding to a distinct acoustic sensor element of the plurality of acoustic sensor elements, the operations further comprising: detecting the adventitious feature based on applying one or more of the plurality of logical channels to a disease pattern definitions logic.

9. The system of claim 1, further comprising: a sensor array apparatus comprising a wearable article, wherein at least one acoustic sensor element of the plurality of acoustic sensor elements is incorporated with the wearable article.

10. The system of claim 1, the operations further comprising: causing the user interface to display an indication of a position on a patient corresponding to detection of the adventitious feature.

11. A method for multiple sensor based acoustic respiratory monitoring, the method comprising: receiving acoustic measurement data derived from one or more breathing sounds as captured by a sensor array comprising a plurality of acoustic sensor elements; generating a plurality of logical channels based on the acoustic measurement data; detecting an adventitious feature in the one or more breathing sounds using the plurality of logical channels; and causing a display of a user interface comprising an indication of an abnormal respiratory sound in response to detecting the adventitious feature.

12. The system of claim 1 wherein each logical channel of the plurality of logical channels carries a stream of acoustic measurement data corresponding to a distinct acoustic sensor element of the plurality of acoustic sensor elements.

13. The method of claim 11, further comprising: causing the user interface to display at least one of: a location of at least one of the plurality of acoustic sensor elements corresponding to the adventitious feature; and trending information computed at least in part from historical acoustic measurement data obtained from a patient acoustic measurement record.

14. The method of claim 11, further comprising: determining a classification of the adventitious feature based on applying one or more of the plurality of logical channels to a disease pattern definitions logic; and wherein the indication of the abnormal respiratory sound includes the classification of the adventitious feature.

15. An acoustic respiratory monitoring system, the system comprising: a sensor array apparatus comprising a sensor array that includes a plurality of acoustic sensor elements; one or more processors coupled to a memory, the one or more processors to perform acoustic data processing operations comprising: processing a plurality of logical channels that carry acoustic measurement data derived from one or more breathing sounds as captured by the plurality of acoustic sensor elements, wherein each logical channel of the plurality of logical channels carries a stream of acoustic measurement data corresponding to an acoustic sensor element of the plurality of acoustic sensor elements; detecting an adventitious feature in the one or more breathing sounds using the stream of acoustic measurement data from one or more logical channels of the plurality of logical channels; and causing a human machine interface to display an indication of an abnormal respiratory sound in response to detecting the adventitious feature.

16. The system of claim 15, wherein the sensor array apparatus further comprises a wearable article, wherein at least one acoustic sensor element of the plurality of acoustic sensor elements is incorporated with the wearable article.
